(19) United States
(12) Patent Application Publication
(10) Pub. No.: US 2023/0408208 A1
(43) Pub. Date: Dec. 21, 2023

(54) DISPOSABLE HEATING ELEMENT ASSEMBLY

(71) Applicant: Schawbel Technologies LLC, Burlington, MA (US)

(72) Inventors: Charles Earl Lundy, JR., Birmingham, AL (US); Thomas John Williams Bayes, Rothwell (GB); Veronica M. Zsolcsak, Newburyport, MA (US); Samuel John Huzzard, Corby (GB); Harold Howlett, Gulfport, MS (US)

(21) Appl. No.: 18/143,729

(22) Filed: May 5, 2023

Related U.S. Application Data
(60) Provisional application No. 63/403,226, filed on Sep. 1, 2022.
(60) Provisional application No. 63/350,163, filed on Jun. 8, 2022.

(51) Int. Cl. F28D 20/00; F24V 30/00

(52) U.S. Cl. CPC F28D 20/003; F28D 2020/0026; F24V 30/00

(57) ABSTRACT

The invention is a portable heater assembly for retaining a disposable heating element within and further providing a means for controlling heat output therefrom.

### CROSS-REFERENCE TO RELATED APPLICATIONS

This application claims priority to, and the benefit of, U.S. Provisional Application No. 63/350,163, filed on Jun. 8, 2022, and U.S. Provisional Application No. 63/403,226, filed on Sep. 1, 2022, the content of each of which is incorporated by reference herein in its entirety.

### TECHNICAL FIELD

The disclosure relates generally to portable warming devices, more particularly, to an assembly for retaining a disposable heating element within and further providing a means for controlling heat output therefrom.

### BACKGROUND

Portable warmers are used by consumers to heat their hands or feet in cold weather. They are popular with participants in winter sports and activities. Portable warmers may be used, as an example, within gloves, boots, or medical bandages; they may be round or circular, ovoid, rectangular, or another shape.

Some portable warmers are electrical and can be battery powered, either rechargeable or single use; such warmers utilize the warming of an electrical coil to release heat to the user. Commercially available examples include the TOMUS-UNI magnetic rechargeable hand warmer or the OCOOPA electric rechargeable pocket heater.

Other portable warmers use chemical reactions for heat generation. Some chemical reactions utilize phase change of the materials to generate heat and may exist in a vacuum without the exchange of ambient air; such materials may be sodium acetate in supersaturated solution with a metal disc that acts as a nucleation center to form and cause crystallization of the solution, releasing heat. Reuse of the materials is possible by adding heat back to the solution such as by placing in hot water, then allowing to cool slowly to room temperature, whereupon the reaction can be restarted by activating the metal disc again. Commercially available examples include HOTSNAPZ reusable hand warmers or CLIK CLAK reusable heat packs.

Still other portable warmers often depend on oxidation reactions that release heat. When exposed to the air, the active agent is activated and begins its chemical process. Usually, this occurs until the ingredient is used up, meaning that such portable warmers are single use. Such portable handwarmers may be any of a commercially available portable warmer utilizing oxygen to activate the fuel therein. Heat is generated in such a portable warmer by an oxidation reaction of the active ingredient in the warmer. This may be a reaction with iron to create ferric oxide, or rust. Other agents may be present to facilitate the exothermic, or heat producing reaction, such as cellulose, activated charcoal, vermiculite, and salt. Commercially available examples include HOTHANDS hand warmers or TUNDRAS hot hand warmers.

A drawback of such portable warmers is the fact that they are essentially single use and generate heat in an all-or-nothing fashion. More specifically, once a consumer opens the packaging, the contents of the warmer are exposed to the surrounding environment and heat generation begins immediately until the reaction completes (i.e., once the ingredients taking part in the exothermic reaction are consumed). As such, there is little control over the heat output of disposable portable warmers.

### SUMMARY

The present invention recognizes the drawbacks of portable warmers, particularly those that rely on oxidation reactions. To address such drawbacks, the present invention provides a portable warmer assembly that generally includes a container configured to control heat generation of disposable portable warmers. In particular, the container is configured to receive and retain a thermally oxidative composition within, such as a conventional oxidation-based disposable heating element configured to provide a thermochemical, exothermic reaction of iron and oxygen in the presence of activated charcoal and sodium chloride. The container further includes one or more ports having respective airflow regulation members for controlling the flow of ambient air from an exterior portion of the container, through the associated port, and into the interior cavity of the container to thereby control exposure of the thermally oxidative composition to oxygen within the ambient air.

Accordingly, the container of the present invention provides the unique ability to regulate the amount of oxygen that can react with the iron/activated carbon material inside the product, and, as a result, the temperature and heat time can be adjusted to the consumer's preference.

In one aspect, the present invention provides a portable heater assembly, which generally includes a container configured to receive and retain a thermally oxidative composition within. The container further includes one or more ports having respective airflow regulation members for controlling the flow of ambient air from an exterior portion of the container, through the associated port, and into the interior cavity of the container to thereby control exposure of the thermally oxidative composition to oxygen within the ambient air.

In some embodiments, the container is configured to receive and retain one or more oxidation-based disposable heating elements within. Accordingly, during use, a consumer can purchase the disposable heating elements separately and place such disposable heating elements into the container, at which point the container is configured to be sealed upon receipt of the one or more oxidation-based disposable heating elements within. The seal is an airtight seal to thereby prevent, or postpone the thermochemical, exothermic reaction. For example, in some embodiments, the container may include an adhesive layer configured to provide an airtight seal and enclose the disposable element within. In other embodiments, the container may be sealed via at least one of application of heat thereto, electrostatic forces, and magnetic forces.

In other embodiments, the container may be prefilled with a thermally oxidative composition and the interior cavity is sealed. For example, the oxidative composition may generally be configured to provide a thermochemical, exothermic reaction of iron and oxygen in the presence of activated charcoal and sodium chloride. Accordingly, the container is sealed during the manufacturing process upon receipt of the thermally oxidative composition within.

The airflow regulation member is configured to transition between at least a fully closed configuration, in which ambient air is prevented from flowing through the respective port and a fully open configuration, in which ambient air is allowed to flow through the respective port. In some embodiments, the airflow regulation member is configured to transition to a plurality of positions between the fully closed configuration and the fully open configuration.

In one example, the airflow regulation member may include a tab member configured to transition between at least a fully closed configuration, in which the respective port is completely covered via the tab member and ambient air is blocked from entering the interior cavity of the chamber, and a fully open configuration, in which the respective port is completely unobstructed and ambient air is permitted to freely enter the interior cavity, and a plurality of positions therebetween to thereby uncover an associated portion of the respective port and permit an associated amount of ambient air to enter the interior cavity.

In some embodiments, the tab member may be resealable. For example, the tab member may be adhered to a perimeter of a respective port via a resealable adhesive. Accordingly, a consumer can open a tab member to a desired position to achieve a certain amount of heat generation and then reseal the tab member over the port to reduce heat generation output. Yet still, in other embodiments, the tab member may be single use and is not resealable.

In some embodiments, the airflow regulation member comprises a valve. The valve may include, but is not limited to, a needle valve, flap valve, a butterfly valve, a solenoid valve, ball valve, a slide valve, a thermal actuator, and an electrothermal actuator.

In some embodiments, the airflow regulation member is configured to be manually manipulated between the fully closed configuration and the fully open configuration. Yet still, in other embodiments, the airflow regulation member may be configured to transition between the fully closed and fully open configurations via operation of a motor-assisted mechanism. For example, the motor-assisted mechanism may include, but is not limited to, a solenoid, a motorized screw, and a motorized gearbox.

The container may be made from one or more layers of a material or combination of materials. While the container may be made from a single layer of material, the container may include multiple layers of a material or combination of materials.

For example, the container may include one or more layers comprising at least one of laminated thermoplastics and/or non-woven films, woven materials, injection molded thermoplastic materials, molded silicone materials, and a metal sheet or film.

In some embodiments, the container comprises one or more layers of a film or non-woven material selected from the group consisting of polyethylene terephthalate, polyethylene, polypropylene, polyolefin copolymers, polyamide (such as nylons), polyvinyl dichloride, polyurethane, ethylene vinyl acetate, polyacrylates, polyvinyl difluoride, polystyrene copolymers, and copolymers of any thereof In other embodiments, the container comprises one or more layers of woven materials comprised of single fiber material or a combination of fiber materials selected from the group consisting of cotton, polyester, nylon, cellulosic-based fibers including rayon, viscose, bamboo, lyocell, polyester fibers, acrylate fibers, polyurethane fibers and blends made from these fibers, wool, linen, silk, and other plant or animal-based products. Yet still, in some embodiments, the container comprises one or more layers of a foam material or a lamination of foam with woven or non-woven materials selected from the group consisting of polyurethane, neoprene, chloroprene, EVA copolymers, polyethylene, polyethylene copolymer, butylene, butylene/styrene copolymer, natural latex, polysiloxane, and other foam materials.

The assembly further includes an outer pouch member configured to receive and enclose the container within. The outer pouch member comprises a breathable material. For example, the outer pouch member comprises a knitted structure of synthetic fibers, natural fibers, or a combination thereof. In some embodiments, the outer pouch member comprises a three-dimensional (3D) spacer fabric material. The 3D spacer fabric material comprises at least three layers comprises of a first surface, a second surface, and a layer of vertical filler yarns positioned therebetween and configured to provide varying levels of thickness for volume and cushioning.

The outer pouch member, including the container filled with the thermally oxidative composition within, is configured to be used in conjunction with a garment or device. For example, the outer pouch member is configured to be received within an associated pocket or compartment formed within a garment selected from the group consisting of a glove, jacket, belt, socks, shoes, shirt, pants, underwear, hat or cap, and slippers. In one example, the outer pouch member is configured to be fitted within an associated pocket formed within a full or partial shoe insole.

In some embodiments, the outer pouch member, including the container filled with the thermally oxidative composition within, is configured to be received within an associated pocket or compartment formed within a pillow.

In some embodiments, the outer pouch member, including the container filled with the thermally oxidative composition within, is configured to be fitted within an associated pocket or compartment of a brace or wrap for a body part selected from the group consisting of an ankle, knee, back, hip, wrist, neck, shoulder, and back.

In some embodiments, the outer pouch member, including the container filled with the thermally oxidative composition within, is configured to be fitted within an associated pocket or compartment of a wound care bandage.

### BRIEF DESCRIPTION OF THE DRAWINGS

FIG. 1A is a perspective view of an exemplary embodiment of a container of a portable heater assembly consistent with the present disclosure.

FIG. 1B is a perspective view showing placement of a thermally oxidative composition into the container of the portable heater assembly of FIG. 1A.

FIG. 1C is a perspective view of a sealed container (in which the thermally oxidative composition is sealed within the container).

FIG. 2 is a perspective view of the sealed container showing placement of tabs over ports of the container for controlling influx of air into the container.

FIGS. 3A, 3B, and 3C are perspective views of the sealed container illustrating removing of tabs to thereby increase the flow of ambient air from an exterior portion of the container, through the associated port, and into the interior cavity of the container to thereby control exposure of the thermally oxidative composition to oxygen within the ambient air.

FIG. 4A illustrates placement of the sealed container into an outer pouch member of the portable heater assembly.

FIG. 4B is a perspective view of the outer pouch member containing the sealed container within.

FIG. 5 shows an exemplary 3D spacer material, partly in section.

FIG. 6 shows a glove and placement of a portable heater assembly (i.e., an outer pouch member enclosing a sealed container with the thermally oxidative composition provided within) into the glove.

FIGS. 7A and 7B show views of an insole for footwear that includes a pocket or compartment for receipt of a portable heater assembly consistent with the present disclosure.

FIG. 8 shows placement of a portable heater assembly on a knee brace.

### DETAILED DESCRIPTION

By way of overview, the present invention is directed to portable warming devices. In particular, the present invention provides a portable heater assembly for retaining a disposable heating element within and further providing a means for controlling heat output therefrom based on controlling exposure of the heat element to oxygen.

In particular, the present invention provides a portable warmer assembly that generally includes a container configured to control heat generation of disposable portable warmers. The container is configured to receive and retain a thermally oxidative composition within, such as a conventional oxidation-based disposable heating element configured to provide a thermochemical, exothermic reaction of iron and oxygen in the presence of activated charcoal and sodium chloride. The container further includes and one or more ports having respective airflow regulation members for controlling the flow of ambient air from an exterior portion of the container, through the associated port, and into the interior cavity of the container to thereby control exposure of the thermally oxidative composition to oxygen within the ambient air.

In particular, the container is designed to receive the thermally oxidative composition, such as a conventional oxidation-based disposable heating element, to be sealed airtight, and have one or more ports that can be opened and closed to regulate the amount of air interacting with the heating element. Sealing all the ports stops the chemical reaction once all the remaining air in the pouch is used up, which may keep the heating element ready for future use.

Accordingly, the container of the present invention provides the unique ability to regulate the amount of oxygen that can react with the iron/activated carbon material inside the product, and, as a result, the temperature and heat time can be adjusted to the consumer's preference.

As will be described in greater herein, the portable heater assembly of the present invention includes a container (for receiving and sealing a thermally oxidative composition within and controlling exposure to oxygen to thereby control heat output) and an outer pouch member for receiving a sealed container within and providing a measure of protection during use (i.e., preventing direct contact between container and a person's skin).

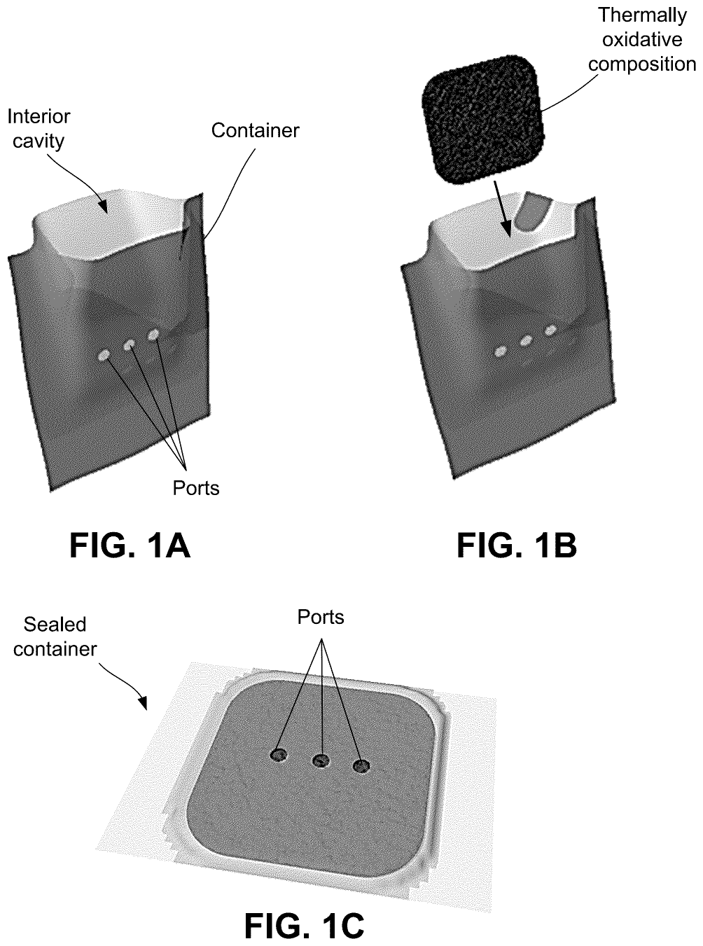

FIG. 1A is a perspective view of an exemplary embodiment of a container of a portable heater assembly consistent with the present disclosure. As shown, the container includes an interior cavity shaped and/or sized to receive and retain a thermally oxidative composition within. The container further includes one or more ports having respective airflow regulation members for controlling the flow of ambient air from an exterior portion of the container, through the associated port, and into the interior cavity of the container.

FIG. 1B is a perspective view showing placement of a thermally oxidative composition into the container of the portable heater assembly of FIG. 1A. In some embodiments, the container is configured to receive and retain one or more oxidation-based disposable heating elements within. Accordingly, during use, a consumer can purchase the disposable heating elements separately and place such disposable heating elements into the container, at which point the container is configured to be sealed upon receipt of the one or more oxidation-based disposable heating elements within.

FIG. 1C is a perspective view of a sealed container (in which the thermally oxidative composition is sealed within the container). The seal is an airtight seal to thereby prevent, or postpone the thermochemical, exothermic reaction. For example, in some embodiments, the container may include an adhesive layer configured to provide an airtight seal and enclose the disposable element within. In other embodiments, the container may be sealed via at least one of application of heat thereto, electrostatic forces, and magnetic forces.

In other embodiments, the container may be prefilled with a thermally oxidative composition and the interior cavity is sealed. For example, the oxidative composition may generally be configured to provide a thermochemical, exothermic reaction of iron and oxygen in the presence of activated charcoal and sodium chloride. Accordingly, the container is sealed during the manufacturing process upon receipt of the thermally oxidative composition within.

The container may be made from one or more layers of a material or combination of materials. While the container may be made from a single layer of material, the container may include multiple layers of a material or combination of materials.

For example, the container may include one or more layers comprising at least one of laminated thermoplastics and/or non-woven films, woven materials, injection molded thermoplastic materials, molded silicone materials, and a metal sheet or film.

In some embodiments, the container comprises one or more layers of a film or non-woven material selected from the group consisting of polyethylene terephthalate, polyethylene, polypropylene, polyolefin copolymers, polyamide (such as nylons), polyvinyl dichloride, polyurethane, ethylene vinyl acetate, polyacrylates, polyvinyl difluoride, polystyrene copolymers, and copolymers of any thereof In other embodiments, the container comprises one or more layers of woven materials comprised of single fiber material or a combination of fiber materials selected from the group consisting of cotton, polyester, nylon, cellulosic-based fibers including rayon, viscose, bamboo, lyocell, polyester fibers, acrylate fibers, polyurethane fibers and blends made from these fibers, wool, linen, silk, and other plant or animal-based products. Yet still, in some embodiments, the container comprises one or more layers of a foam material or a lamination of foam with woven or non-woven materials selected from the group consisting of polyurethane, neoprene, chloroprene, EVA copolymers, polyethylene, polyethylene copolymer, butylene, butylene/styrene copolymer, natural latex, polysiloxane, and other foam materials.

The length and width dimensions of the pouch are from 0.1 cm to 25 cm independently. Each dimension may be less than 0.1 cm, 0.1 cm, 0.2 cm, 0.3 cm, 0.4 cm, 0.5 cm, 0.6 cm, 0.7 cm, 0.8 cm, 0.9 cm, 1 cm, 2 cm, 3 cm, 4 cm, 5 cm, 6 cm, 7 cm, 8 cm, 9 cm, 10 cm, 11 cm, 12 cm, 13 cm, 14 cm, 15 cm, 16 cm, 17 cm, 18 cm, 19 cm, 20 cm, 21 cm, 22 cm, 23 cm, 24 cm, 25 cm, greater than 25 cm, or a measurement between any of the above. The depth dimension of the pouch may be from .1 cm to 10 cm. The depth dimension may be less than 0.1 cm, 0.1 cm, 0.2 cm, 0.3 cm, 0.4 cm, 0.5 cm, 0.6 cm, 0.7 cm, 0.8 cm, 0.9 cm, 1 cm, 2 cm, 3 cm, 4 cm, 5 cm, 6 cm, 7 cm, 8 cm, 9 cm, 10 cm, greater than 10 cm, or a measurement between any of the above. Furthermore, the container may include any particular shape, including, but not limited to a generally rectangular, circular, or ovoid shape, or another shape.

Manipulation of the airflow regulation members associated with the given ports allows for controlling exposure of the thermally oxidative composition to oxygen. In particular, heat generated from the thermally oxidative composition can generally be controlled by preventing or allowing air to flow into the interior cavity of the container via the airflow regulation members. For example, in some embodiments, the generation of heat can be started, stopped, and, in some instances, be restarted. Furthermore, the total heat output can be increased/decreased based on manipulation of the overall air flow through the ports, as will be described in greater detail herein.

In the illustrated embodiment, the container has multiple ports (i.e., perforations formed in the container material) that control the rate that oxygen can reach the thermally oxidative composition in the container. In some embodiments, each of the ports are the same size and shape. However, it should be noted that one or more ports may have different shapes and/or sizes. By providing multiple ports, the amount of control over oxygen influx is greatly improved. For example, a consumer may desire to have a small amount of heat to be generated. Accordingly, the consumer can control oxygen influx by manipulating only a single airflow regulation member of a single port while the other ports and associated airflow regulation members remain unchanged. Accordingly, only a single port is exposed and allowing air to flow into the interior cavity, which may result in a small amount of heat generation, as compared to manipulating all airflow regulation members to thereby allow air to flow into the interior cavity via all available ports.

In some embodiments, the one or more ports may be sized differently to let different rates of air into the pouch. In other embodiments, all of the ports are the same size. Accordingly, in such an embodiment, opening all the ports would let the most air in, thereby exposing the thermally oxidative material to the most oxygen influx, thereby resulting in the most heat generation output and using the most fuel. However, opening of just a single port and/or opening of the smallest of ports, would result in the least oxygen influx, thereby resulting in the least amount of heat generation (with at least one port open) using the least amount of fuel (with at least one port open). Each port may independently have a size of larger than 1 gauge, 1 gauge, 2 gauge, 3 gauge, 4 gauge, 5 gauge, 6 gauge, 7 gauge, 8 gauge, 9 gauge, 10 gauge, 11 gauge, 12 gauge, 13 gauge, 14 gauge, 15 gauge, 16 gauge, 17 gauge, 18 gauge, 19 gauge, 20 gauge, 21 gauge, 22 gauge, 23 gauge, 24 gauge, 25 gauge, 26 gauge, 27 gauge, 28 gauge, 29 gauge, 30 gauge, or smaller sizes.

The airflow regulation member is configured to transition between at least a fully closed configuration, in which ambient air is prevented from flowing through the respective port and a fully open configuration, in which ambient air is allowed to flow through the respective port. In some embodiments, the airflow regulation member is configured to transition to a plurality of positions between the fully closed configuration and the fully open configuration.

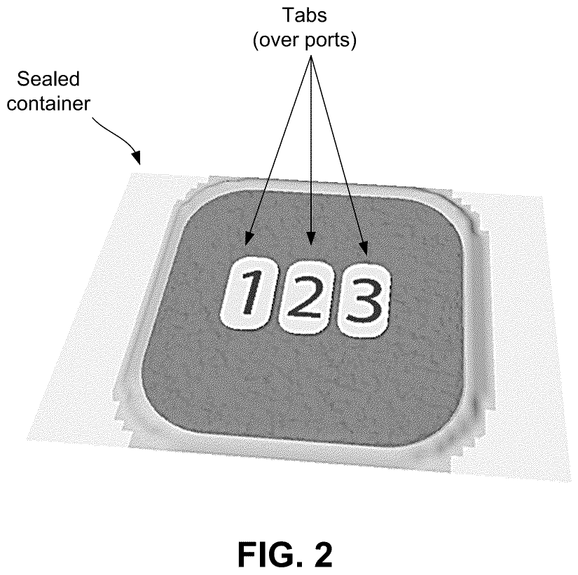

FIG. 2 is a perspective view of the sealed container showing placement of tabs over ports of the container for controlling influx of air into the container. As shown, in one example, the airflow regulation member may include a tab member configured to transition between at least a fully closed configuration, in which the respective port is completely covered via the tab member and ambient air is blocked from entering the interior cavity of the chamber, and a fully open configuration, in which the respective port is completely unobstructed and ambient air is permitted to freely enter the interior cavity, and a plurality of positions therebetween to thereby uncover an associated portion of the respective port and permit an associated amount of ambient air to enter the interior cavity.

In some embodiments, the tab member may be resealable. For example, the tab member may be adhered to a perimeter of a respective port via a resealable adhesive. Accordingly, a consumer can open a tab member to a desired position to achieve a certain amount of heat generation and then reseal the tab member over the port to reduce and even stop heat generation output. Accordingly, the consumer can resume and restart heat generation by simply pulling away one or more of the tabs. Yet still, in other embodiments, the tab member may be single use and is not resealable.

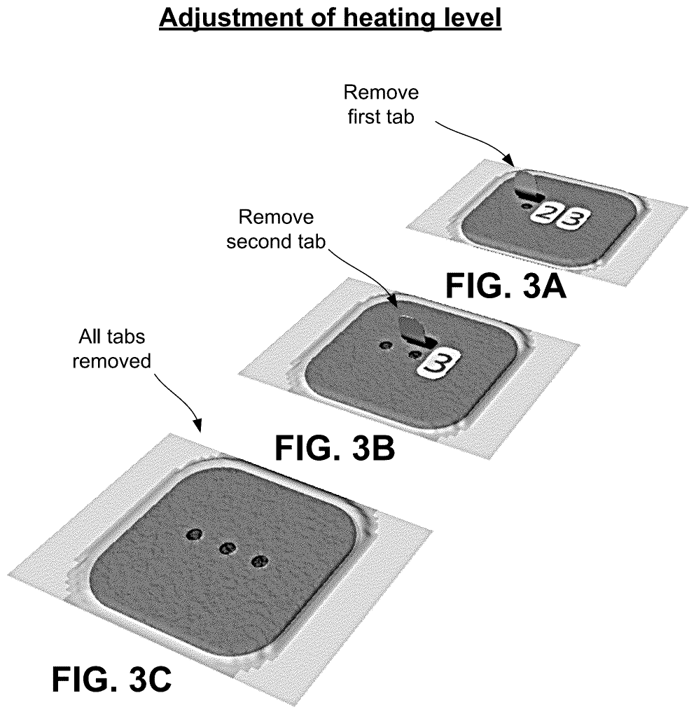

FIGS. 3A, 3B, and 3C are perspective views of the sealed container illustrating removing of tabs to thereby increase the flow of ambient air from an exterior portion of the container, through the associated port, and into the interior cavity of the container to thereby control exposure of the thermally oxidative composition to oxygen within the ambient air. For example, a consumer can adjust the level of associated heat by removing one or more tablets. In particular, a consumer can remove a first tab member (while the other tab members remain in place and in a fully closed configuration), which may then result in a low level heat setting. Upon removing a second tab member, the heat setting may increase to a medium level heat setting (due to the increased oxygen influx as a result of removal of both the first and second tab members). Finally, a consumer can remove the third tab member, thereby resulting in unobstructed air flow paths through all ports, resulting in a high level heat setting.

While the airflow regulation members have been illustrated as tab members, it should be noted that, in other embodiments, the airflow regulation member may include a valve. The valve may include, but is not limited to, a needle valve, flap valve, a butterfly valve, a solenoid valve, ball valve, a slide valve, a thermal actuator, and an electrothermal actuator. It should be noted that the range of sizes of the gate valve/flap gate is from 1.0 mm to 100 mm at maximum axis length and the flap valve/flap gate may have multiple shapes including circular, oval, triangular, square, rectangular, polygonal. It should be noted that the slide valve may have a maximum opening between 1.0 mm to 100 mm at maximum axis length, and may have a maximum opening greater than 100 mm at maximum axis length.

In some embodiments, the airflow regulation member is configured to be manually manipulated between the fully closed configuration and the fully open configuration. Yet still, in other embodiments, the airflow regulation member may be configured to transition between the fully closed and fully open configurations via operation of a motor-assisted mechanism. For example, the motor-assisted mechanism may include, but is not limited to, a solenoid, a motorized screw, and a motorized gearbox.

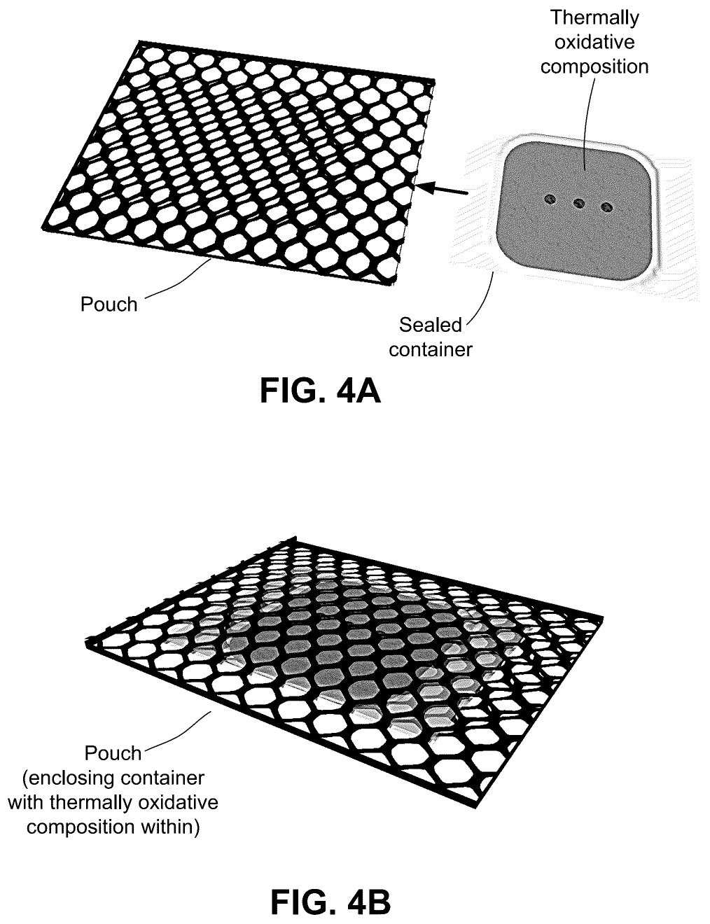

Once a heating level is set to a desired level (by manipulating one or more airflow regulation members), the container can be placed within a protective outer pouch member. FIG. 4A illustrates placement of the sealed container into an outer pouch member of the portable heater assembly. FIG. 4B is a perspective view of the outer pouch member containing the sealed container within.

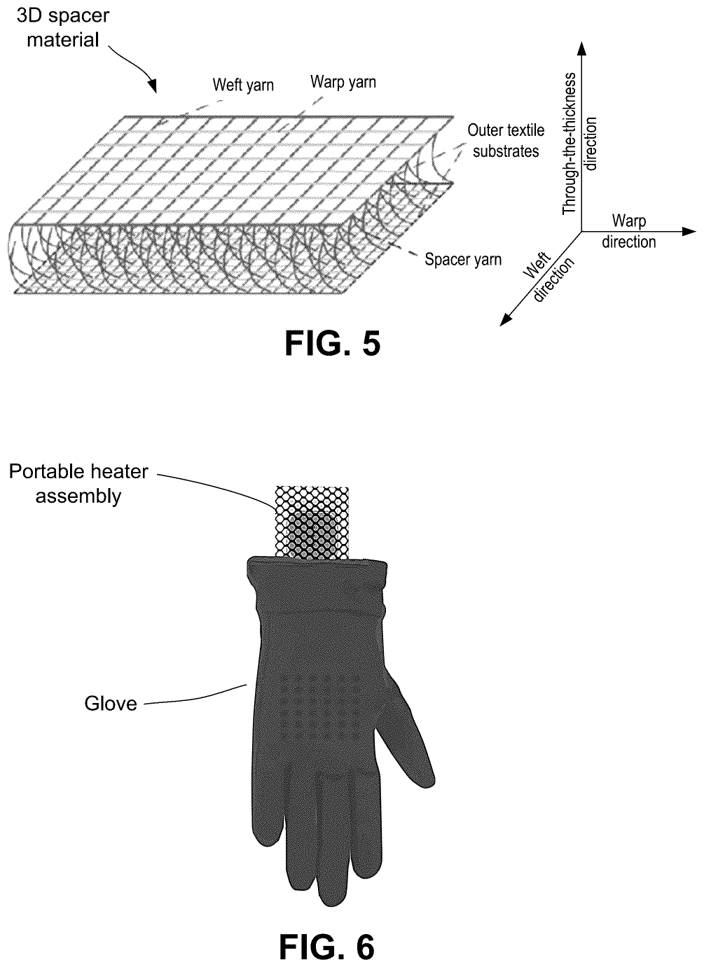

As illustrated, the outer pouch member is configured to receive and enclose the container within. The outer pouch member comprises a breathable material. For example, the outer pouch member comprises a knitted structure of synthetic fibers, natural fibers, or a combination thereof. In some embodiments, the outer pouch member comprises a three-dimensional (3D) spacer fabric material. FIG. 5 shows an exemplary 3D spacer material, partly in section. The 3D spacer fabric material comprises at least three layers comprises of a first surface, a second surface, and a layer of vertical filler yarns positioned therebetween and configured to provide varying levels of thickness for volume and cushioning.

The outer pouch member, including the container filled with the thermally oxidative composition within, is configured to be used in conjunction with a garment or device. For example, the outer pouch member is configured to be received within an associated pocket or compartment formed within a garment selected from the group consisting of a glove, jacket, belt, socks, shoes, shirt, pants, underwear, hat or cap, and slippers.

FIG. 6 shows a glove and placement of a portable heater assembly (i.e., an outer pouch member enclosing a sealed container with the thermally oxidative composition provided within) into the glove.

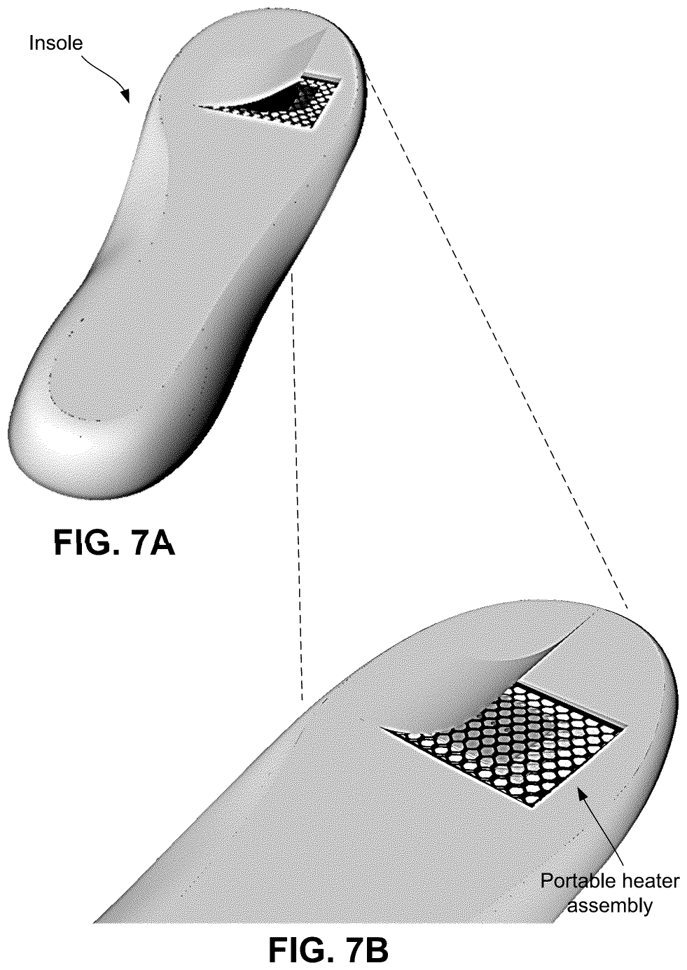

FIGS. 7A and 7B show views of an insole for footwear that includes a pocket or compartment for receipt of a portable heater assembly consistent with the present disclosure.

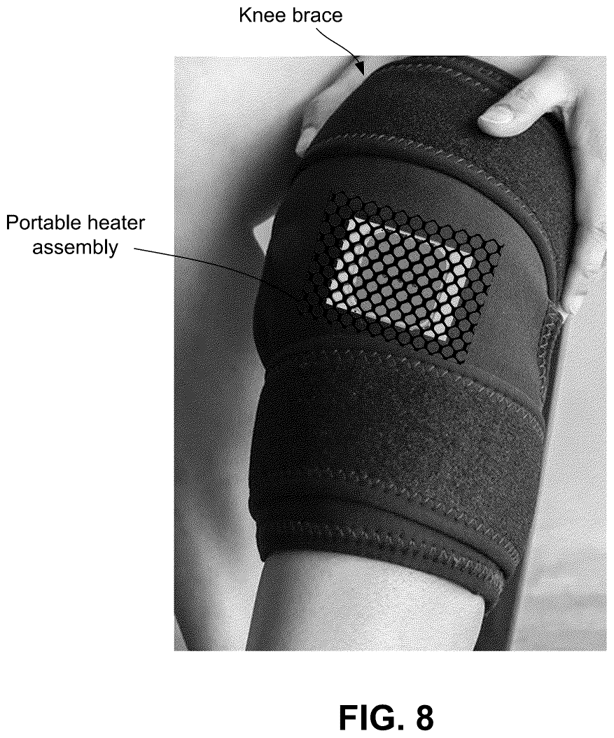

In some embodiments, the outer pouch member, including the container filled with the thermally oxidative composition within, is configured to be fitted within an associated pocket or compartment of a brace or wrap for a body part selected from the group consisting of an ankle, knee, back, hip, wrist, neck, shoulder, and back. FIG. 8 shows placement of a portable heater assembly on a knee brace.

In some embodiments, the outer pouch member, including the container filled with the thermally oxidative composition within, is configured to be received within an associated pocket or compartment formed within a pillow.

In some embodiments, the outer pouch member, including the container filled with the thermally oxidative composition within, is configured to be fitted within an associated pocket or compartment of a wound care bandage.

Reference throughout this specification to “one embodiment” or “an embodiment” means that a particular feature, structure, or characteristic described in connection with the embodiment is included in at least one embodiment. Thus, appearances of the phrases “in one embodiment” or “in an embodiment” in various places throughout this specification are not necessarily all referring to the same embodiment. Furthermore, the particular features, structures, or characteristics may be combined in any suitable manner in one or more embodiments.

The terms and expressions which have been employed herein are used as terms of description and not of limitation, and there is no intention, in the use of such terms and expressions, of excluding any equivalents of the features shown and described (or portions thereof), and it is recognized that various modifications are possible within the scope of the claims. Accordingly, the claims are intended to cover all such equivalents.

### Incorporation by Reference

References and citations to other documents, such as patents, patent applications, patent publications, journals, books, papers, web contents, have been made throughout this disclosure. All such documents are hereby incorporated herein by reference in their entirety for all purposes.

### Equivalents

Various modifications of the invention and many further embodiments thereof, in addition to those shown and described herein, will become apparent to those skilled in the art from the full contents of this document, including references to the scientific and patent literature cited herein. The subject matter herein contains important information, exemplification and guidance that can be adapted to the practice of this invention in its various embodiments and equivalents thereof.

1. A portable warmer assembly comprising:
a container comprising:
an interior cavity shaped and/or sized to receive and retain a thermally oxidative composition within; and
one or more ports having respective airflow regulation members for controlling the flow of ambient air from an exterior portion of the container, through the associated port, and into the interior cavity of the container to thereby control exposure of the thermally oxidative composition to oxygen within the ambient air.

2. The assembly of claim 1, wherein the container is configured to receive and retain one or more oxidation-based disposable heating elements within, each of the oxidation-based disposable heating elements comprising a thermally oxidative composition.

3. The assembly of claim 2, wherein the container is configured to be sealed upon receipt of the one or more oxidation-based disposable heating elements within the interior cavity.

4. The assembly of claim 3, wherein the container comprises an adhesive layer configured to provide an airtight seal with respect to the interior cavity and the one or more oxidation-based disposable heating elements within.

5. The assembly of claim 1, wherein the container is prefilled with a thermally oxidative composition and the interior cavity is sealed.

6. The assembly of claim 5, wherein the oxidative composition is configured to provide a thermochemical, exothermic reaction of iron and oxygen in the presence of activated charcoal and sodium chloride.

7. The assembly of claim 1, wherein the airflow regulation member is configured to transition between at least:
a fully closed configuration, in which ambient air is prevented from flowing through the respective port; and
a fully open configuration, in which ambient air is allowed to flow through the respective port.

8. The assembly of claim 7, wherein the airflow regulation member comprises a tab member configured to transition between at least a fully closed configuration, in which the respective port is completely covered via the tab member and ambient air is blocked from entering the interior cavity of the chamber, and a fully open configuration, in which the respective port is completely unobstructed and ambient air is permitted to freely enter the interior cavity, and a plurality of positions therebetween to thereby uncover an associated portion of the respective port and permit an associated amount of ambient air to enter the interior cavity.

9. The assembly of claim 7, wherein the airflow regulation member comprises a valve selected from the group consisting of a needle valve, flap valve, a butterfly valve, a solenoid valve, ball valve, a slide valve, a thermal actuator, and an electrothermal actuator.

10. The assembly of claim 7, wherein the airflow regulation member is configured to be manually manipulated between the fully closed configuration and the fully open configuration.

11. The assembly of claim 7, wherein the airflow regulation member transitions between the fully closed and fully open configurations via operation of a motor-assisted mechanism selected from the group consisting of a solenoid, a motorized screw, and a motorized gearbox.

12. The assembly of claim 1, wherein the container comprises one or more layers of a material or combination of materials, the one or more layers comprising at least one of laminated thermoplastics and/or non-woven films, woven materials, injection molded thermoplastic materials, molded silicone materials, and metal sheets or films.

13. The assembly of claim 12, wherein the container comprises one or more layers of a film or non-woven material selected from the group consisting of polyethylene terephthalate, polyethylene, polypropylene, polyolefin copolymers, polyamide (such as nylons), polyvinyl dichloride, polyurethane, ethylene vinyl acetate, polyacrylates, polyvinyl difluoride, polystyrene copolymers, and copolymers of any thereof.

14. The assembly of claim 12, wherein the container comprises one or more layers of woven materials comprised of single fiber material or a combination of fiber materials selected from the group consisting of cotton, polyester, nylon, cellulosic-based fibers including rayon, viscose, bamboo, lyocell, polyester fibers, acrylate fibers, polyurethane fibers and blends made from these fibers, wool, linen, silk, and other plant or animal-based products.

15. The assembly of claim 12, wherein the container comprises one or more layers of a foam material or a lamination of foam with woven or non-woven materials selected from the group consisting of polyurethane, neoprene, chloroprene, EVA copolymers, polyethylene, polyethylene copolymer, butylene, butylene/styrene copolymer, natural latex, polysiloxane, and other foam materials.

16. The assembly of claim 1, further comprising an outer pouch member configured to receive and enclose the container within.

17. The assembly of claim 16, wherein the outer pouch member comprises a breathable material.

18. The assembly of claim 16, wherein the outer pouch member comprises a knitted structure of synthetic fibers, natural fibers, or a combination thereof

19. The assembly of claim 16, wherein the outer pouch member comprises a three-dimensional (3D) spacer fabric material.

20. The assembly of claim 19, wherein 3D spacer fabric material comprises at least three layers comprises of a first surface, a second surface, and a layer of vertical filler yarns positioned therebetween and configured to provide varying levels of thickness for volume and cushioning.
